(19) United States
(12) Patent Application Publication
(10) Pub. No.: US 2017/0281374 A1
(43) Pub. Date: Oct. 5, 2017

(54) STENT

(71) Applicant: TERUMO KABUSHIKI KAISHA, Tokyo (JP)

(72) Inventors: Tomoya KOMATSU, Hadano-city (JP); Haruhiko KAMIJO, Hadano-city (JP); Takashi KUMAZAWA, Fujinomiya-city (JP)

(73) Assignee: TERUMO KABUSHIKI KAISHA, Tokyo (JP)

(21) Appl. No.: 15/627,576

(22) Filed: Jun. 20, 2017

Related U.S. Application Data
(63) Continuation of application No. PCT/JP2015/081654, filed on Nov. 10, 2015.

(30) Foreign Application Priority Data
Dec. 24, 2014 (JP) 2014-261064

(51) Int. Cl. A61F 2/88; A61F 2/915; A61F 2/89

(52) U.S. Cl. CPC A61F 2/88; A61F 2/89; A61F 2/915; A61F 2002/91591; A61F 2002/041

(57) ABSTRACT

A stent Includes a stent body formed by a strut. The stent body is cylindrically-shaped and extends in an axial direction. The stent body includes a plurality of helical portions and an annular portion. The strut is helically-shaped along the axial direction to form the plurality of helical portions and the strut is annularly-shaped in the circumferential direction to form the annular portion. Each of the plurality of helical portions has a distal end point and a proximal end point. The plurality of helical portions include a first helical portion and a second helical portion adjacent to the first helical portion in the axial direction. The annular portion is disposed between the first and second helical portions in the axial direction. At least one of the distal and proximal end points of the plurality of helical portions is directly connected to the annular portion.

### CROSS-REFERENCES TO RELATED APPLICATIONS

This application is a continuation of International Application No. PCT/JP2015/081654 filed on Nov. 10, 2015, and claims priority to Japanese Patent Application No. 2014-261064 filed on Dec. 24, 2014, the entire content of both of which is incorporated herein by reference.

### TECHNICAL FIELD

The present invention generally relates to a stent serving as a medical device.

### BACKGROUND DISCUSSION

A stent is a medical device used to treat various diseases caused by a stenosed or occluded lumen of a blood vessel. A stent is used for securing a cavity by widening the stenosed or occluded site. A stent is known in which a strut is formed in a helical shape along an axial direction, for example, as disclosed in Japanese Patent Application Publication No. 2009-522022.

A type of the stent in which the strut is formed in the helical shape in this way includes a balloon expandable stent which is not provided with a self-expandable function and which is expanded by a balloon, or a self-expandable stent which is expanded using a self-elastic deformation force.

### SUMMARY

The strut described above is formed in the helical shape along the axial direction. Accordingly, one end side is separated from the other end side (i.e., at the opposite end) of the strut along the axial direction. A stent having this configuration is less likely to maintain a shape particularly after the diameter of the stent expands. Consequently, in a case where the stent is deformed to reduce the diameter by itself while attempting to restore the original shape of the stent, it is difficult to restrain/control the deformation. That is, the stent formed in a helical shape along the axial direction as described above cannot sufficiently restrain a contraction force (diameter reduction force) generated due to the deformation with the diameter expansion, and it is difficult to decrease a recoil rate of the stent.

The stent discloses here addresses the above-described problem, and possesses a relatively lower recoil rate than the strut formed in the helical shape along the axial direction.

The stent disclosed in this application has a stent body that is formed into a cylindrical shape by a strut. The stent body includes a plurality of helical portions in which the strut is formed in a helical shape along an axial direction, and an annular portion that is disposed between the helical portions adjacent to each other in the axial direction. The annular portion of the strut is formed in an annular shape along the circumferential direction.

The annular portion in which one end side and the other end side (i.e., at the opposite end) are endlessly connected to each other is more likely to maintain its shape in a radial direction K than the helical portion described above in which one end side and the other end side are separated from each other. Therefore, after the stent body expands the diameter, the annular portion can restrain the helical portion from being deformed when the stent body attempts to reduce the diameter so as to restore the original shape. That is, the stent can restrain a contraction force (diameter reduction force) generated due to the deformation with the diameter expansion, and it is possible to considerably decrease a recoil rate.

### BRIEF DESCRIPTION OF THE DRAWINGS

FIGS. 1(A) and 1(B) are views illustrating an embodiment of a stent. FIG. 1(A) is a perspective view of the stent, and FIG. 1(B) is a perspective view illustrating only one side of the stent based on line 1B-1B in FIG. 1(A) by dividing the integrally formed stent in FIG. 1(A) into each configuration along an axial direction.

FIGS. 2(A) and 2(B) are views illustrating a stent in which the stent illustrated in FIG. 1(A) is schematically configured. FIG. 2(A) is a perspective view of the schematically configured stent, and FIG. 2(B) is a side view of a portion of the schematically configured stent.

FIGS. 3(A) and 3(B) are views illustrating the stent shown in FIG. 1(A), FIG. 3(A) is a development view of the stent, and FIG. 3(B) is a view illustrating an enlarged portion of a broken line portion labeled 3B in FIG. 3(A).

FIG. 4 is a development diagram illustrating an enlarged main portion of the stent shown in FIG. 1(A).

FIG. 5 is a development view illustrating an enlarged main portion of a stent according to a modification example.

### DETAILED DESCRIPTION

Set forth below with reference to the accompanying drawings is a detailed description of embodiments of a stent representing examples of the inventive stent disclosed here. The following description does not limit the technical scope or the meaning of terms described in the scope of the appended claims. Dimensional proportions in the drawings are exaggerated and different from actual dimensional proportions for convenience of description. In all of the drawings, a stent is illustrated in a state where the stent is cut out from an original metal pipe when the stent is manufactured (i.e., a state before the stent is crimped on, for example, a delivery catheter).

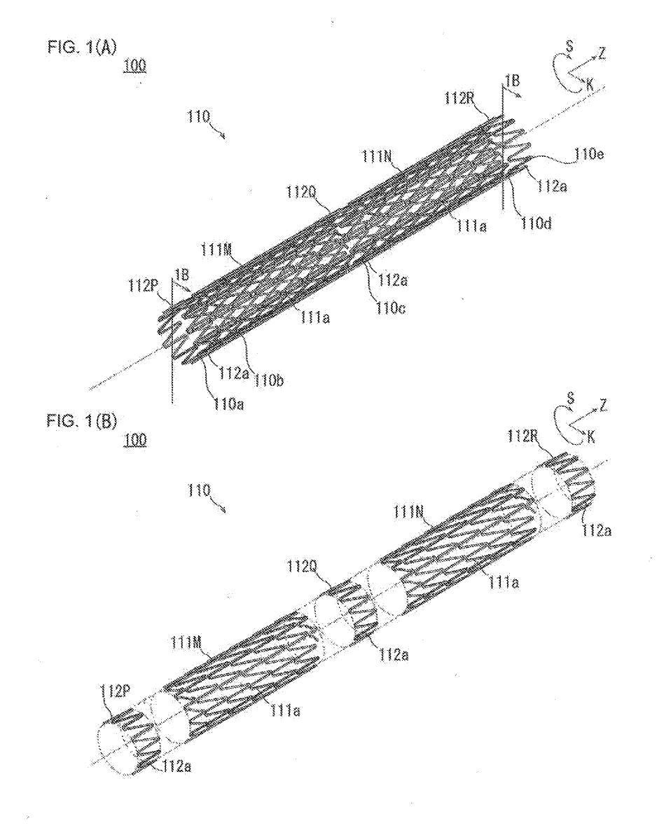

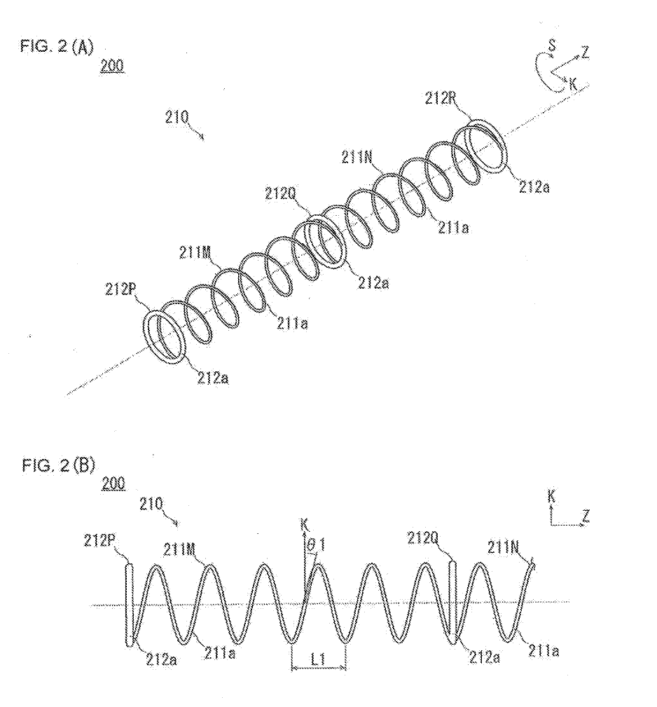

FIGS. 1(A), 1(B), 3(A), 3(B), and 4 are views for describing a configuration and an operation of a stent 100 according to an embodiment of the disclosed stent. FIGS. 2(A) and 2(B) are views for describing a configuration and an operation of a stent 200 in which the stent 100 is schematically illustrated. In the description herein, a longitudinal direction (lateral direction in FIG. 1(A)) of the stent 100 is referred to as an axial direction Z. A direction along a concentric circle around the axial direction Z as a central axis is referred to as a circumferential direction S. A direction along a radial direction around the axial direction Z as the central axis is referred to as a radial direction K.

As illustrated in FIG. 1(A), the stent 100 has a stent body 110 in which a strut (e.g., linear configuration element) having an integrally connected coil shape is formed. As a whole, the stent 100 possesses a substantially cylindrical outer shape having a predetermined length in the axial direction Z. The stent 100 is caused to indwell a lumen (for example, a blood vessel, a biliary duct, a bronchial tube, an esophagus, other gastrointestinal tracts, and a urethra) inside a living body and is used for treating a stenosed or occluded site by widening a cavity of the lumen.

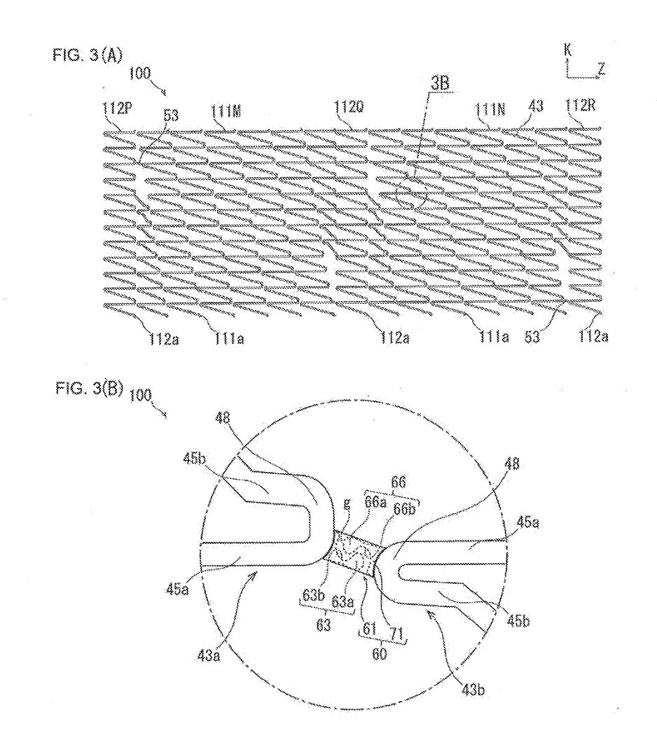

The stent body 110 integrally includes helical portions 111 (111M, 111N) and annular portions 112 (112P, 112Q, and 112R) as illustrated in FIGS. 1(A) and 3(A). The helical portions 111 (111M, 111N) and the annular portions 112 (112P, 112Q, and 112R) which are integrally formed are illustrated in FIG. 1(B) in a state where all of these are separated from each other along the axial direction Z to facilitate an understanding of the stent body 110. The helical portions 111M and 111N each possess the same shape, and a strut 111a is formed in a helical shape along the axial direction Z. Specifically, a plurality of the helical portions 111M and 111N respectively extend in a helical shape along the circumferential direction S of the stent body 110 while being turned back in a wavelike manner in the axial direction Z of the stent body 110 (i.e., each helical portion 111M and 111N possesses a wavy-shape including apexes/bends where the helical portion 111M and 111N changes direction in the axial direction Z).

The annular portions 112P, 112Q, and 112R each possess the same shape, and a strut 112a is formed in an annular shape along the circumferential direction S. Specifically, the annular portion 112P, 112Q, and 112R are each formed in an annular shape along the circumferential direction S while alternately protruding and curving toward a proximal side 110b and a distal side 110d along the axial direction Z. In this manner, the annular portion 112P, 112Q, and 112R are configured to have an endless shape (i.e., the annular portions 112P, 112Q, 112R are non-helical, or in other words, the annular portions 112P, 112Q, 112R are endless loops without a beginning or ending point). The annular portion 112Q is disposed between helical portions 111M and 111N that are adjacent to each other along the axial direction Z. The annular portion 112Q is thus located in a central portion 110c of the stent body 110. An end point of the helical portions 111M and 111N is directly connected to the annular portion 112Q. The annular portion 112P is disposed adjacent to the helical portion 111M so that the annular portion 112P is located in the proximal portion 110a on the proximal side 110b of the stent body 110. An end point of the helical portion 111M is directly connected to the annular portion 112P. The annular portion 112R is disposed adjacent to the helical portion 111N so that the annular portion 112R is located in a distal portion 110e on the distal side 110d of the stent body 110. An end point of the helical portion 111N is directly connected to the annular portion 112R.

FIGS. 2(A)-2(B) illustrate a stent 200 in which the stent 100 is schematically configured in order to facilitate an understanding of the stent 100. As illustrated in FIG. 2(A), an annular portion 212P, a helical portion 211M, an annular portion 212Q, a helical portion 211N, and an annular portion 212R are integrally formed in a stent body 210 of the stent 200. The annular portion 212P, helical portion 211M, annular portion 212Q, helical portion 211N, and annular portion 212R are integrally formed in this order from the proximal side to the distal side along the axial direction Z of the stent body 210. The annular portion 212P, helical portion 211M, annular portion 212Q, helical portion 211N, and annular portion 212R of the stent body 210 respectively correspond to the annular portion 112P, helical portion 111M, annular portion 112Q, helical portion 111N, and annular portion 112R of the stent body 110.

As shown by the schematic illustration in FIG. 2(B), the helical portion 211 (for example, helical portion 211M) of the stent body 210 is configured to include struts 211a which have a constant interval L1 along the axial direction Z (i.e., the struts 211a are equally spaced relative to one another) and which intersect the radial direction K at a helical angle θ1 (i.e., extend to form the helical angle θ1 relative to the radial direction K as shown in FIG. 2(B)). A connection portion 60 (to be described later in reference to FIGS. 3(A) and 3(B)) connects portions of the struts 211a to each other at locations adjacent along the axial direction Z. The struts 211a are thus constrained along the axial direction Z by the connection portion 60 (connector). Accordingly, if the diameter of the stent body 210 expands, the struts 211a extend outward in the radial direction while the interval L1 between the struts 211a with respect to the axial direction Z remains constant. That is, the helical portion 211 is constrained along the axial direction Z by the connection portion 60. In this manner, the helical portion 211 is changed and twisted so that the helical angle θ1 of the struts 211a becomes larger. However, the stent 200 includes the struts 212a of the endlessly formed annular portion 212 that sufficiently restrain the helical portion 211 from being deformed.

As illustrated in FIG. 3(A), a link portion 63 connects the helical portion 111 and the annular portion 112 to each other in the stent body 110 of the stent 100. As illustrated in FIG. 3(B), the helical portion 111 included in the strut 111a has a pair of linear portions 45a and 45b extending by tilting at a predetermined angle with respect to the axial direction Z of the stent body 110, and a curved portion (turned-back portion) 48 disposed between the pair of linear portions 45a and 45b (i.e., the curved portion 48 is directly connected to, and extends between, the linear portions 45a and 45b). The linear portions 45a and 45b and the curved portion 48 are repeatedly formed along a predetermined length, thereby configuring one helical portion 111. A plurality of helical portions 111 are disposed side by side in series in the axial direction Z of the stent body 110, thereby allowing the overall stent 100 to configure one helix. The number of the helical portions 111 and the number of the curved portions 48 is not particularly limited.

As illustrated in FIG. 3(B), the connection portion 60 has a connection structure 61 formed integrally with the helical portion 111 of the strut 111a and a connection member 71 configured to include a biodegradable material. The connection structure 61 is formed by adding a predetermined shape to a pair of helical portions 43a and 43b arranged adjacent so as to face each other in the axial direction Z (e.g., the helical portions 43a and 43b may each have an extension that facilitates connecting the helical portions 43a and 43b together). In the example illustrated in FIG. 3(B), the connection structure 61 includes a first engagement portion 63 formed in one helical portion (hereinafter, referred to as a “first helical portion”) 43a, and a second engagement portion 66 formed in the other helical portion (hereinafter, referred to as a “second helical portion”) 43b. The first engagement portion 63 and the second engagement portion 66 are engaged with (hooked to) each other to mechanically connect the helical portions 43a and 43b to each other.

The first engagement portion 63 has a first protruding portion 63a and a first housing portion 63b. The first protruding portion 63a is formed to protrude toward the second helical portion side from the curved portion 48. The first housing portion 63b is formed to be recessed in a concave shape between the first protruding portion 63a and the curved portion 48. The second engagement portion 66 similarly has a second protruding portion 66a formed to protrude toward the first helical portion side from the curved portion 48 and a second housing portion 66b formed to be recessed in a concave shape between the second protruding portion 66a and the curved portion 48.

The first protruding portion 63a included in the first engagement portion 63 is formed so that a shape of the distal portion is curved (i.e., the distal end of the first protruding portion 63a is curved as shown in FIG. 3(B)). The second housing portion 66b included in the second engagement portion 66 is formed so that the first protruding portion 63a can be housed therein. The second protruding portion 66a included in the second engagement portion 66 is formed so that a shape of the distal portion is curved. The first housing portion 63b included in the first engagement portion 63 is formed so that the second protruding portion 66a can be housed therein. When the first protruding portion 63a is housed inside the second housing portion 66b and the second protruding portion 66a is housed inside the first housing portion 63b, the first helical portion 43a and the adjacent second helical portion 43b are connected to one another via the first engagement portion 63 and the second engagement portion 66.

The respective protruding portions 63a and 66a can be arranged to form a gap g between the respective housing portions 63b and 66b. The respective protruding portions 63a and 66a can also be arranged to partially come into contact with the respective housing portions 63b and 66b. In addition, the respective engagement portions 63 and 66 can be arranged so that the engagement portions 63 and 66 partially or entirely overlap each other in a region along the circumferential direction S and/or the axial direction Z of the stent 100 as illustrated in FIG. 3(B). It is thus possible to strongly hook the respective engagement portions 63 and 66 to each other, and it is possible to stably maintain a connection between the first engagement portion 63 and the second engagement portion 66. It is also possible to arrange the respective protruding portions 63a and 66a to face each other in a direction tilting with respect to the axial direction Z as illustrated in FIG. 3(B) (e.g., the connection between the protruding portions 63a and 66a is titled/sloped along the extent of the connection in the axial direction Z). If both of the protruding portions 63a and 66a are arranged in this way, for example, a distance between the respective protruding portions 63a and 66a decreases when a tensile force is applied in a direction to separate the first helical portion from the second helical portion along the axial direction Z. The protruding portions 63a and 66a are thus attached to each other. In this manner, the first engagement portion 63 and the second engagement portion 66 are strongly hooked to each other. Accordingly, it is possible to more reliably maintain a connection state between the helical portions 43a and 43b.

The connection member 71 covers a surface of the connection structure 61 and fills portions between the respective protruding portions 63a and 66a and the respective housing portions 63b and 66b. A configuration can be adopted in which a concave portion is formed or a through-hole penetrating both front and back surfaces is formed on the surface of the respective engagement portions 63 and 66. The connection member 71 may thus fill the concave portion or the through-hole. According to this configuration, it is possible to improve adhesion (i.e., the adhesive force) of the connection member 71 adhering to the connection structure 61.

Since the stent 100 includes the helical portion 111, the stent 100 is flexible. Therefore, the stent's ability to follow the deformation of the lumen (followability) is improved. The connection member 71 formed of the biodegradable material having a relatively strong physical property (i.e., the biodegradable material applies a relatively strong adhesion/boding force to connect the helical portions 111 to each other) is disposed in a portion connecting the helical portions 111 to each other. Accordingly, the stent body 110 can be provided with desirable rigidity. While satisfactory followability to follow the deformation of the lumen is ensured, an expansion holding force can be improved when the stent 100 is caused to indwell the lumen. The indwelling connection member 71 degrades after a predetermined period elapses so that the connecting force of the connection portion 60 is weakened. When this degradation occurs, flexibility of the stent 100 is further improved. Accordingly, the followability of the stent to follow the deformation of the lumen is further improved. Therefore, in an initial stage of the indwelling period, a desired expansion holding force is achieved, and after the predetermined period elapses from the indwelling and the connection member 71 degrades, improved flexibility is achieved. The stent 100 thus becomes relatively excellent in regard to invasiveness and a treatment effect. In addition, the annular portion 112P and the annular portion 112R (which are disposed in both end portions of the stent body 110) maintain a predetermined expansion holding force regardless of the degradation of the connection member 71. Therefore, it is possible to apply a sufficient expansion holding force to the lumen from both end portions of the stent body 110 even after the connection member 71 is degraded. Accordingly, it is possible to suitably prevent the stent 100 from being misaligned after the stent 100 indwells.

It is preferable to provide one or more connection portions 60 for each one of the helical portions 43 (one unit of the helical portion 111 in the circumferential direction). However, the number of connection portions 60 is not particularly limited. The structure of the connection portion 60, and the form of the connection structure 61 and the connection member 71 which are included in the connection portion 60, is not limited to the above-described configurations. The structure and the form can be appropriately changed. For example, a shape of the respective engagement portions 63 and 66 included in the connection structure 61 can be different than the shapes discussed above as long as the mechanical connection can be created. The connection portion 60 can be configured to change the connecting force without interposing the connection member 71 therebetween. For example, it is possible to employ a fragile portion (which is more likely to be broken than other portions) in a portion of the connection structure 61. The fragile portion is broken after a predetermined period elapses in a state where the stent 100 indwells. In this manner, the connection structure 61 can oscillate (is movable).

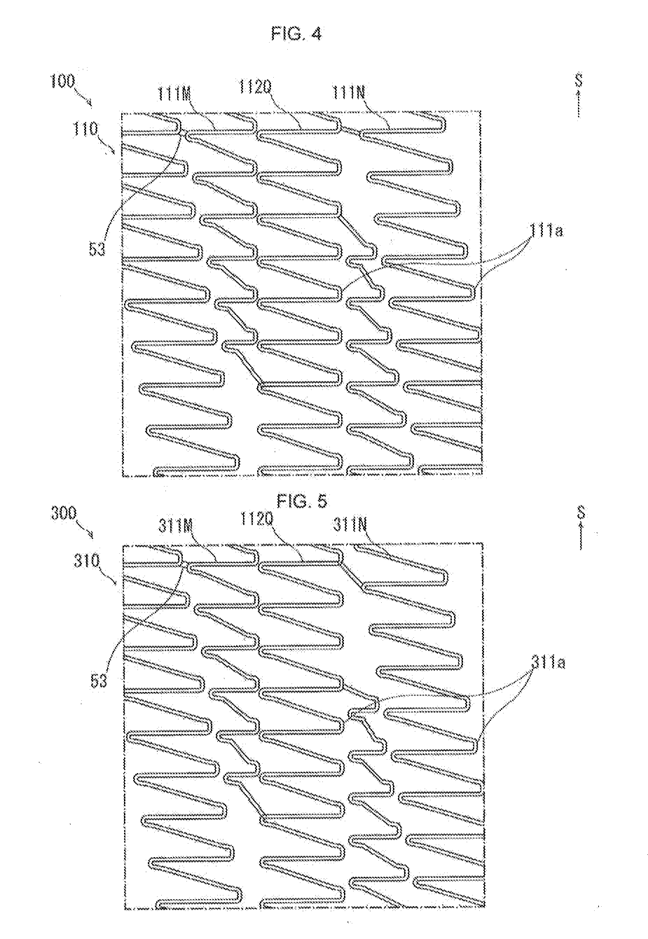

As illustrated in FIG. 4, the stent 100 has patterns of the helical shapes of the helical portions 111 (111M and 111N) adjacent to each other across the annular portion 112 that are coincident with each other along the circumferential direction S. Specifically, positions of the struts 111a of the helical portion 111M and the helical portion 111N (which are adjacent to each other along the axial direction Z) are aligned with each other along the circumferential direction S. An end point of the helical portion 111M connected to the annular portion 112P and an end point of the helical portion 111N connected to the annular portion 112Q are aligned with each other along the circumferential direction S. Furthermore, an end point of the helical portion 111M connected to the annular portion 112Q and an end point of the helical portion 111N connected to the annular portion 112R are aligned with each other along the circumferential direction S. The end point of the helical portion 111M is connected to the annular portion 112Q at a first point, the helical portion 111N is connected to the annular portion 112Q at a second point, and the first and second points may be shifted (i.e., misaligned) relative to one another in the circumferential direction as shown in FIGS. 3(A) and 4(A). As further illustrated in FIG. 3(A) and 4(A), the first helically-wound strut 111M includes a distal portion and a proximal portion, the second helically-wound strut 111N includes a distal portion and a proximal portion, and the distal and proximal portions of the first and second helically-wound struts each include struts (or strut portions) possessing lengths gradually changing in the axial direction (e.g., the axial length of the struts decreases in a tapering manner in the circumferential direction as the struts get closer to the end point where the helical portion 111M or 111N is connected to the annular portion 112R, 112Q or 112P as shown in FIG. 3(A)).

When the stent 100 is configured to serve as a balloon expandable stent, a known metal may be appropriately selected for a material of the stent body 110. The metal for the stent body 110 may be stainless steel which is a non-biodegradable metal material, a cobalt-based alloy such as a cobalt-chromium alloy, or an elastic metal such as a platinum-chromium alloy. On the other hand, when the stent 100 is configured to serve as a self-expandable stent, a known super-elastic alloy may be appropriately selected.

The connection member 71 is formed of a biodegradable material such as a biodegradable polymer material or a biodegradable metal material. The biodegradable polymer material is preferably a biodegradable synthetic polymer material such as polylactic acid, polyglycolic acid, lactic acid-glycolic acid copolymer, polycaprolactone, lactic acid-caprolactone copolymer, glycolic acid-caprolactone copolymer, and poly-γ-glutamic acid, or a biodegradable natural polymer material such as cellulose and collagen. Magnesium or zinc may, for example, be used for the biodegradable metal material.

A drug coated layer containing a drug can be formed in the stent 100. For example, the drug coated layer can be disposed on an entire outer surface on a side coming into contact with the lumen of the living body or on a portion of the outer surface. The drug coated layer may contain a drug carrier for carrying the drug or may be configured to contain only the drug without the drug carrier. For example, a thickness of the drug coated layer is 1 to 300 μm, and preferably 3 to 30 μm.

Examples of the drug contained in the drug coated layer are anticancer drugs, immunosuppressive drugs, antibiotics, anti-rheumatic drugs, anti-thrombotic drugs, HMG-CoA reductase inhibitors, insulin resistance improving drugs, ACE inhibitors, calcium antagonists, anti-hyperlipidemic drugs, integrin inhibitors, anti-allergic drugs, anti-oxidants, GP IIb/IIIa antagonists, retinoids, flavonoids, carotenoids, lipid improving drugs, DNA synthesis inhibitors, tyrosine kinase inhibitors, antiplatelet drugs, anti-inflammatory drugs, biologically-derived materials, interferon, and nitric oxide production-promoting substances.

When the stent 100 is configured to treat a stenosed site in the blood vessel, it is preferable that the drug coated layer contains paclitaxel, docetaxel, sirolimus, and/or everolimus. It is more preferable that the drug coated layer contains sirolimus or paclitaxel.

It is preferable that the drug carrier is polymer material, and more preferably a biodegradable polymer material which degrades inside a living body. After the stent 100 is caused to indwell in the lumen of the living body, the biodegradable polymer material carrying the drug degrades. The drug is released to restrain restenosis at the stent indwelling site. It is possible to use the same materials as those discussed above regarding the connection member 71 for the biodegradable polymer material.

As described above, the stent body 110 includes a plurality of helical portions 111 (in which the strut 111a is formed in a helical shape along the axial direction Z) and an annular portion 112 disposed between the helical portions 111 adjacent to each other in the axial direction Z. The strut 112a is formed in an annular shape along the circumferential direction S to form the annular portion 112. According to this configuration, the annular portion 112 (in which one end side and the other end side are endlessly connected to each other) is more likely to maintain its shape in the radial direction K than the helical portion 111 (in which one end side and the other end side are separated from each other). Therefore, after the stent 100 expands and the diameter of the stent body 110 increases, the annular portion 112 can restrain the helical portion 111 from deforming when the stent body 110 attempts to reduce the diameter to restore the original shape. That is, the stent 100 can restrain a contraction force (diameter reduction force) generated due to the deformation with the diameter expansion, and it is possible to considerably decrease a recoil rate.

The stent body 110 further has the annular portions 112 (112P and 112R) on at least one side of the proximal side 110b and the distal side 110d along the axial direction Z. Accordingly, at least one side of the proximal side 110b and the distal side 110d can also help restrain the deformation of the helical portion 111 which attempts to reduce the diameter of the stent body 110. Therefore, it is possible to further decrease the recoil rate of the stent 100.

At least one annular portion 112 (112Q) of the stent body 110 is disposed in a central portion 110c between the proximal portion 110a and the distal portion 110e in the axial direction Z. The at least one annular portion 112Q helps make it possible to sufficiently restrain the deformation of the central portion 110c which is most likely to be distorted within a range along the axial direction Z of the stent body 110. Therefore, it is possible to effectively decrease the recoil rate of the stent 100.

The stent 100 further has the connection portion 60 that connects at least one location of windings of the helical portion 111 in the axial direction Z of the stent body 110. The connection portion 60 decreases the connecting force after a predetermined period elapses when the stent body 110 indwells in the living body. Here, the helical portion 111 is constrained along the axial direction Z by the connection portion 60. Accordingly, when the stent body 110 expands the diameter, the struts 111a extend outward in the radial direction of the struts 111a while the interval L1 between the struts 111a with respect to the axial direction Z remains constant. That is, the helical portion 111 is constrained along the axial direction Z by the connection portion 60. The helical portion 111 is thus changed and twisted so that the helical angle θ1 of the struts 111a becomes larger. As a result, the helical portion 111 attempts to be untwisted by reducing the diameter. Even according to the configuration in which the stent 100 has the connection portion 60, the annular portion 112 can sufficiently restrain the deformation of the helical portion 111. Therefore, it is possible to effectively decrease the recoil rate.

The connection portion 60 has the connection member 71 configured to include the biodegradable material. Accordingly, it is possible to decrease the connecting force as time elapses in accordance with the degradation of the biodegradable material. Therefore, it is possible to achieve satisfactory followability inside the lumen.

The annular portions 112 are formed in an annular shape along the circumferential direction S (i.e., the annular portions 112 are non-helical) while alternately protruding and curving the strut 112a toward the proximal side 110b and the distal side 110d along the axial direction Z (i.e., the annular portions 112 have a wavy-shape in the circumferential direction S as shown in FIG. 1(B)). Accordingly, the curved portion is widened along the circumferential direction S, thereby also enabling the stent 100 to expand the diameter outward in the radial direction. That is, it is possible to sufficiently expand the diameter of the stent 100 acting in the radially outward direction (which is opposite to the radially inward direction) while the annular portion 112 effectively decreases the recoil rate of the stent 100 acting in the radially inward direction.

### Modification Example

A stent 300 according to a modification example will be described with reference to FIG. 5.

The stent 300 has a configuration different from that of the stent 100 according to the embodiment discussed above in that the arrangement patterns of the helical shapes of adjacent helical portions 311 are different from each other (e.g., the patterns created by the helical shapes of adjacent helical portions 311 are different from one another in the circumferential and/or axial directions). In the modification example according to the embodiment illustrated in FIG. 5, the same reference numerals will be given to the components which have the same configuration as discussed above, and repeated description will be omitted.

FIG. 5 is a view illustrating a configuration of the stent 300. The operation of the stent 300 according to the modification example will be described.

Helical portions 311 (311M and 311N) adjacent to each other across the annular portion 112 have different arrangement patterns of the helical shapes along the circumferential direction S. The helical portion 311M is spaced along the axial direction Z from the helical portion 311N. The positions of struts 311a of the helical portion 311M and are shifted from the struts 311a of the adjacent helical portion 311N by an angle corresponding to a half pitch along the circumferential direction S. The helical portions 311 (311M and 311N) adjacent to each other across the annular portion 112 may have different shapes. When the relative dimensions or the shapes of the curved portions 48 are different from each other, the helical portions 311 (311M and 311N) possess different flexibility characteristics.

The helical portions 311 (311M and 311N) adjacent to each other across the annular portion 112 have mutually different arrangement patterns of the respective helical shapes as discussed above. Accordingly, the annular portions 112 that have different flexibility characteristics restrain mutual contraction forces (diameter reduction forces). Therefore, it is possible to effectively decrease the recoil rate.

The helical portions 311 (311M and 311N) have different arrangement patterns of the helical shapes along the circumferential direction S. Accordingly, the mutual contraction forces (diameter reduction forces) are applied to the respective annular portions 112 of the stent 100 so as to be restrained along the circumferential direction S. It is thus possible to considerably decrease the recoil rate. In particular, for example, when the stent 300 is caused to indwell the blood vessel located in the vicinity of the heart, the recoil subsequently occurring due to the blood vessel twisting and deforming in response to the pulsation of the heart can be relieved along the circumferential direction S.

The stent disclosed in this application has been described above in reference to the embodiments illustrated in FIGS. 1(A)-5. However, the stent may be appropriately modified based on the scope of the appended claims and is not limited to only the configurations described or illustrated.

For example, at least one or more annular portions of the stent may be disposed at a position separated from (spaced apart from) the end point of the distal portion and the proximal portion in a region between the distal portion and the proximal portion along the axial direction Z. For example, the stent may include four or more annular portions so that two annular portions are respectively disposed at each of the ends of the distal portion and the proximal portion along the axial direction Z and two annular portions are disposed therebetween. In addition, the two helical portions adjacent to one another on either side of a central annular portion may possess a helical direction (winding direction) that are opposite one another (e.g., the two helical portions may be wound in opposite directions).

The stent may be a balloon expandable stent which can be caused to indwell the living body in such a way that a balloon catheter is used as a delivery catheter and the stent is deformed with diameter expansion of the dilated (expanded) balloon. Alternatively, the stent may be a self-expandable stent which indwells in the living body by using a catheter having a slidable cover sheath as a delivery catheter. The stent itself is deformed with diameter expansion (i.e., self-expands radially outwardly) after the stent is released from the sheath.

The base material of the stent is not limited to those which are configured to include the metal material. As long as the stent is configured to include a material which can be elastically deformed so as to generate the recoil, any base material may be employed. For example, the stent body may be configured to include a biodegradable polymer material.

In addition, a structure, dimension, and shape of each element of the stent disclosed in this application can be appropriately changed. For example, the use of the additional member described in an embodiment can be appropriately omitted, or other additional members which are not particularly described in the embodiment can be appropriately used.

The detailed description above describes a stent. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A stent comprising:
a stent body formed by a strut, the stent body being cylindrically-shaped and extending in an axial direction from a distal end to a proximal end, the stent body possessing a circumferential direction;
the stent body comprising a plurality of helical portions and an annular portion, the strut being helically-shaped along the axial direction to form the plurality of helical portions and the strut being annularly-shaped in the circumferential direction to form the annular portion, each of the plurality of helical portions comprising a distal end point and a proximal end point;
the plurality of helical portions comprising a first helical portion and a second helical portion spaced apart from one another in the axial direction, the annular portion being disposed between the first and second helical portions in the axial direction; and
at least one of the distal and proximal end points of the plurality of helical portions is directly connected to the annular portion.

2. The stent according to claim 1, wherein the annular portion is a first annular portion and the stent body comprises a second annular portion, the second annular portion being located at the proximal end or at the distal end of the stent body along the axial direction.

3. The stent according to claim 2, wherein the first annular portion is disposed in a central portion of the stent body between the proximal end and the distal end in the axial direction.

4. The stent according to claim 1, further comprising:
a connection portion that connects at least one location of windings of the plurality of helical portions in the axial direction of the stent body, the connection portion applying a connection force to connect the at least one location of the windings, and
the connecting force decreasing after a predetermined period elapses when the stent body indwells in a living body.

5. The stent according to claim 4, wherein the connection portion has a connection member that is configured to include a biodegradable material.

6. The stent according to claim 1, wherein the strut is annularly-shaped along the circumferential direction to form the annular portion while the strut also alternately elongates and curves toward the proximal end and the distal end in the axial direction throughout the extent of the annular portion.

7. The stent according to claim 1, wherein the first and second helical portions adjacent to each other on either side of the annular portion have different arrangement patterns of the respective helical shapes.

8. The stent according to claim 7, wherein the first and second helical portions have different arrangement patterns of the helical shapes along the circumferential direction.

9. The stent according to claim 1, wherein
the strut comprises a first helical strut forming the first helical portion, a second helical strut forming the second helical portion, and an annular strut forming the annular portion, and
the first helical strut, the second helical strut, and the annular strut are integrally connected to one another.

10. A stent comprising:
a stent body formed by a strut into a cylindrical shape, the stent body extending in an axial direction from a distal end to a proximal end, the stent body possessing a circumferential direction;
the stent body comprising a plurality of helical portions and an annular portion, the strut being helically-shaped along the axial direction to form the plurality of helical portions and the strut being annularly-shaped in the circumferential direction to form the annular portion;
the plurality of helical portions comprising a first helical portion and a second helical portion adjacent to the first helical portion in the axial direction, the annular portion being between the first and second helical portions in the axial direction; and
the first and second helical portions each possess a helical pattern along the circumferential direction of the stent, the helical pattern of the first helical portion being one of coincident with the helical pattern of the second helical portion along the circumferential direction of the stent or shifted from the helical pattern of the second helical portion by an angle corresponding to a half pitch along the circumferential direction of the stent.

11. A stent comprising:
a cylindrical stent body extending in an axial direction from a distal end to a proximal end, the stent body possessing a circumferential direction;
the stent body comprising a first helically-wound strut, a second helically-wound strut and an annular strut, the first and second helically-wound struts each comprising a distal end point and a proximal end point, each of the first and second helically-wound struts comprising multiple windings in the circumferential direction between the distal end point and the proximal end point, the annular strut being a single winding in the circumferential direction; and
the annular strut being positioned between the first helically-wound strut and the second helically-wound strut in the axial direction, the annular strut being directly connected to the proximal end point of the first helically-wound strut and the distal end point of the second helically-wound strut so that the first helically-wound strut, the annular strut and the second helically-wound strut are integrally connected to one another to form the stent body.

12. The stent according to claim 11, wherein
the stent body further comprises a distal annular strut and a proximal annular strut, the distal annular strut being at the distal end of the stent body and the proximal annular strut being at the proximal end of the stent body.

13. The stent according to claim 12, wherein the distal annular strut is directly connected to the distal end of the first helically-wound strut and the proximal annular strut is directly connected to the proximal end of the second helically-wound strut.

14. The stent according to claim 11, wherein
the stent body possesses a center between the distal end and the proximal end of the stent body, and
the annular strut is at the center of the stent body.

15. The stent according to claim 11, further comprising
a first connection portion connecting two of the multiple windings of the first helically-wound strut to one another by applying a connection force to the two of the multiple windings of the first helically wound strut; and
a second connection portion connecting two of the multiple windings of the second helically-wound strut to one another by applying a connection force to the two of the multiple windings of the second helically wound strut.

18. The stent according to claim 15, wherein
the first and second connection portions each comprise a biodegradable material; and
the biodegradable material degrades to reduce the connection force applied by the first and second connection portions after a predetermined period of time elapses from when the stent is inserted into a living body.

17. The stent according to claim 11, wherein the first and second helically-wound struts each comprise a distal portion and a proximal portion, the distal and proximal portions of the first and second helically-wound struts each comprising struts possessing lengths gradually changing in the axial direction.

18. The stent according to claim 11, wherein the proximal end point of the first helically-wound strut is connected to the annular strut at a first point, the distal end point of the second helically-wound strut is connected to the annular strut at a second point, and the first point is shifted relative to the second point along the circumferential direction of the stent.
